(19) United States
(12) Patent Application Publication
(10) Pub. No.: US 2019/0030076 A1
(43) Pub. Date: Jan. 31, 2019

(54) COMPOSITION COMPRISING CELLS TREATED WITH 15-PGDH INHIBITOR OR CULTURE THEREOF AND USE THEREOF

(71) Applicants: Kyu Boem HAN, Daejeon (KR); EHLBIO CO., LTD., Seoul (KR)

(72) Inventors: Kyu Boem HAN, Daejeon (KR); Hong Ki LEE, Seoul (KR); Seong Hun LEE, Seoul (KR)

(73) Assignees: EHLBIO CO., LTD., Seoul (JP); Kyu Boem HAN, Seoul (KR)

(21) Appl. No.: 15/771,853

(22) PCT Filed: Oct. 27, 2016
(86) PCT No.: PCT/KR2016/012125

§ 371 Date: Oct. 11, 2018

(30) Foreign Application Priority Data
Oct. 27, 2015 (KR) 10-2015-0149142
Oct. 26, 2016 (KR) 10-2016-0139967

(51) Int. Cl. A61K 35/28; C12N 5/0775; A61L 27/38; A61L 27/54; A61P 37/06

(52) U.S. Cl. CPC A61K 35/28; C12N 5/0667; C12N 2501/999; A61L 27/54; A61P 37/06; A61L 27/3834

(57) ABSTRACT

The present invention relates to a composition containing cells, cultured by adding 15-PGDH inhibitor to PGE2-expressing cells, or a culture thereof and a use thereof. More specifically, the present invention relates to: a pharmaceutical composition, containing cells, cultured by adding 15-PGDH inhibitor to PGE2-expressing cells, or a culture thereof, for the prevention or treatment of immunological diseases, inflammatory diseases, or wounds; a method for inhibiting an immune response, an inflammatory response, or wounds in a subject, the method comprising a step for administering the pharmaceutical composition, or the cells or the culture thereof, to the subject; a method for preparing an immunosuppressive agent, an antiinflammatory agent, or a wound healing agent, using the cells or the culture thereof; a method for preparing PGE2, the method comprising a step for adding a 15-PGDH inhibitor to PGE2-expressing cells, followed by culturing; an implant comprising PGE2-expressing cells and a 15-PGDH inhibitor; a method for preparing the implant; a complex comprising PGE2-expressing cells and a 15-PGDH inhibitor; and a culture obtained by adding a 15-PGDH inhibitor to PGE2-expressing cells.

### BACKGROUND OF THE INVENTION

### Field of the Invention

The present invention relates to a composition containing cells, cultured by adding 15-PGDH inhibitor to PGE2-expressing cells, or a culture thereof and a use thereof. More specifically, the present invention relates to: a pharmaceutical composition, containing cells, cultured by adding a 15-PGDH inhibitor to PGE2-expressing cells, or a culture thereof, for the prevention or treatment of immunological diseases, inflammatory diseases, or wounds; a method for inhibiting an immune response, an inflammatory response, or wounds in a subject, the method comprising a step for administering the pharmaceutical composition, or the cells or the culture thereof, to the subject; a method for preparing an immunosuppressive agent, an anti-inflammatory agent, or a wound healing agent, using the cells or the culture thereof; a method for preparing PGE2, the method comprising a step for adding a 15-PGDH inhibitor to PGE2-expressing cells, followed by culturing; an implant comprising PGE2-expressing cells and a 15-PGDH inhibitor; a method for preparing the implant; a complex comprising PGE2-expressing cells and a 15-PGDH inhibitor; and a culture obtained by adding a 15-PGDH inhibitor to PGE2-expressing cells.

### Related Art

Prostaglandin E2 (hereinafter, PGE2) is a compound indicated by PGE2: [(5Z,11(alpha), 13E, 15S)-11, 15-dihydroxy-9-oxo-prosta-5, 13-dien-1-oic acid]) and it is the most widely produced prostaglandin in physiological and pathological environments (Ushikubi F et al., J. Pharmacol. Sci. 83: 279, 2000). Conventionally, PGE2 has been used in the uterine cervix to prepare for birth, and it is being sold in the form of a vaginal suppository with the following trademark names: Cervidil (Forest Laboratories), Prostin E2 (Pfizer Limited), Propess (Ferring Pharmaceuticals), and Glandin (Nabiqasim Pharmaceuticals, Pakistan). Additionally, recently, there has been a growing interest in a novel use of PGE2 as immunosuppressive agents and regulators, and this is because it has been reported that PGE2 plays the role of inhibiting the secretion of cytokines such as interleukin-1 beta and TNF alpha produced by macrophages and inhibiting the differentiation of helper T1 cells (Harris S G et al., Trends Immunol., 23: 144, 2002). Additionally, it has been reported that PGE2 inhibits the production of cytokines such as interleukin-1 beta and IFN-gamma thereby inhibiting the differentiation of T cells in humans and mice (Goodwin J S et al., J. clin. Immunol., 3: 295, 1983). Additionally, since the above experimental results have been reported, there is an ample possibility of using it as an immunomodulator, and thus there has been a need for the development of an economical and simple method of production. Transforming growth factor beta 1 (TGF-β1) is known as an immunosuppressive agent and antiinflammatory agent, and due to the ample possibility of using it as an immunomodulatory as is the case of PGE2, there has been a need for the development of an economical and simple method of its production.

### SUMMARY OF THE INVENTION

Meanwhile, immunosuppressive agents include specific inhibitors that inhibit only the responses and nonspecific inhibitors. Theoretically, specific inhibitors should be excellent, but nonspecific inhibitors are mainly used. Examples of the immunosuppressive agents most commonly used clinically include cyclosporine (Neoral, Cipol A), azathioprine (imuran), and prednisolone (a kind of a steroid). The above three kinds of inhibitors were shown to have less side effects and higher immunosuppressive effects compared to when they were administered in combination. Recently, various kinds of immunosuppressive agents such as FK 506, RATG, OKT3, Cellcept, etc. have been developed and are used currently. These immunosuppressive agents inhibit immunosuppression by inhibiting several steps in the process from antigen stimulation to antibody production, such as antigen phagocytosis by macrophages, antigen recognition by lymphocytes, etc., cell division, cell division of T cells and B cells, antibody production, etc. Most immunosuppressive agents have antitumor activity and this is because these agents inhibit cell division mediated by DNA interference, inhibition of DNA synthesis, etc.

However, these agents have side effects, such as hypertension and nephrotoxicity (deterioration of renal function), and due to high incidence of side effects, there has been a problem in that it is necessary to sufficiently observe the progress when using these agents. Other side effects, although rare, may include tremor, seizure, hepatitis, low flow of bile liquid, increase of uric acid in the blood, decrease in muscle strength, hypertrichosis, gingival hypertrophy, etc. Among the frequently-used inhibitors, azathioprine often inhibits bone marrow function, such as decreased leukocyte levels, anemia, platelet reduction, etc. and may occasionally cause complications showing hair loss, fever, etc. along with pancreatitis, hepatitis, low flow of bile. Prednisolone, one of steroid agents, is the first of the immunosuppressive agents that had been used and it shows the broadest inhibitory effect. Prednisolone promotes appetite, increases shoulder and back muscles, and provides temporary happiness. However, these steroid agents not only promote atherosclerosis but also cause hypertension, gastric ulcer, diabetes, growth inhibition, osteoporosis, cataracts, glaucoma, etc. and thus these drugs must be used with caution.

Allograft, such as organ transplantation and hematopoietic stem cell transplantation, is a medical breakthrough achievement in the 21st century, and is used as a fundamental method for treating ten final diseases including heart failure such as dilated cardiomyopathy, chronic renal failure, intractable blood diseases, etc. However, there is a problem in that immune responses such as rejection of transplanted organs and graft-versus-host-disease (GVHD), which is a fatal complication that occurs after allograft, must be overcome. The technology currently used is a treatment method for controlling the immune responses of T cells in which the immune responses after transplantation are developed as cellular immunity by T cells that recognized allogeneic antigens (Ikehara S, Exp. Hematol., 31: 1142, 2003; First M R, Transplantation, 77: 88, 2004) so as to minimize such immunological responses. That is, the treatment method that controls immune responses by inhibiting the production of interleukin (IL)-2 in T cells using cyclosporin and FK506, which are immunosuppressive agents, is used. However, there has been a need for the development of an immunosuppressive agent that can be used economically without side effects.

Meanwhile, the precise mechanism of action of mesenchymal stem cells on immunomodulatory ability has not yet been elucidated, and only several recent reports on mesenchymal stem cells have been suggested as follows. First, mesenchymal stem cells appear to have an effect of inhibiting antigen presenting cells (APCs). Considering that the degree of immune response changes in proportion to the number of mononuclear cells added during the culturing process under certain conditions, it can be assumed that monocytes are involved in the immunosuppressive effect. Second, mesenchymal stem cells appear to exhibit an immunosuppressive effect by modulating the proliferation rate of T cells. When T cells are cultured with mesenchymal stem cells, as cyclin D2 is inhibited, T cells remain in the G0/G1 phase of the cell cycle without a further increase. Additionally, it has been reported that the proliferative capacity is continuously lowered even when the mesenchymal stem cells are removed (Glennie S et al., Blood, 105: 2821, 2005).

### DETAILED DESCRIPTION OF THE INVENTION

Under these circumstances, the present inventors have made efforts to develop a more effective method for controlling immunity or inflammation using cells. As a result, they have confirmed that when cells are treated with a 15-PGDH inhibitor, PGE2 is significantly overproduced and inhibits immune responses more effectively and thus the 15-PGDH inhibitor is effective in an animal model with atopy or autoimmune disease, thereby completing the present invention. Additionally, the present inventors have confirmed that when cells are treated with a 15-PGDH inhibitor, PGE2 is significantly overproduced and thus the 15-PGDH inhibitor can be used for the treatment of wounds.

As used herein, the term “15-PGDH inhibitor” refers to a material (an antagonist) that inhibits the activity of 15-PGDH. For example, the 15-PGDH inhibitor may be at least one kind selected from the group consisting of cyclooxygenase inhibitors, flavonoids, phytophenolic compounds, and antagonists of peroxisome proliferator-activated receptor gamma (PPAR γ), but the 15-PGDH inhibitor is not limited thereto.

As used herein, the term “cell” includes stem cells having the ability to differentiate into various tissues.

As used herein, the term “prevention” refers to all of the actions that inhibit or delay the occurrence of an immunological disease or inflammatory disease by the administration of the composition according to the present invention, and the term “treatment” refers to all of the actions that improve or advantageously change the symptoms of an immunological disease or inflammatory disease by the administration of the pharmaceutical composition according to the present invention.

As used herein, the term “implant”, being a support that allows to isolate a damaged area from the outside or allows the grafted cell or secreted therapeutic material to stay, refers to a material that can be transplanted into the human body or a mammal. Such an implant includes, without limitation, various materials used in the art, such as biodegradable synthetic polymers, natural materials, etc. as a support for tissue engineering.

The present invention provides a pharmaceutical composition for the prevention or treatment of immunological diseases or inflammatory diseases, containing cells cultured by adding a 15-hydroxyprostaglandin dehydrogenase (15-PGDH) inhibitor to prostaglandin E2 (PGE2)-expressing cells, or a culture thereof.

The present invention provides a method for inhibiting an immune response or inflammatory response in a subject excluding humans, which includes administering, to a subject, cells cultured by adding a 15-PGDH inhibitor to PGE2-expressing cells or a culture thereof.

The present invention provides a method for preparing an immunosuppressive agent or antiinflammatory agent, which includes adding a 15-PGDH inhibitor to PGE2-expressing cells followed by culturing the same.

The present invention provides a method for preparing PGE2, which includes culturing PGE2-expressing cells in a medium, to which a 15-PGDH inhibitor was added, and PGE2 or transforming growth factor beta 1 (TGF-β1) is secreted from these cells during cultivation.

The present invention provides an implant which contains PGE2-expressing cells and a 15-PGDH inhibitor.

The present invention provides a method for preparing an implant which includes culturing by adding a 15-PGDH inhibitor to PGE2-expressing cells.

The present invention provides a complex including PGE2-expressing cells and a 15-PGDH inhibitor.

The present invention provides a culture obtained by adding a 15-PGDH inhibitor to PGE2-expressing cells.

Additionally, the present invention provides a pharmaceutical composition for the prevention or treatment of wounds, containing cells cultured by adding a 15-PGDH inhibitor to PGE2-expressing cells, or a culture thereof.

Additionally, the present invention provides a cosmetic composition containing cells cultured by adding a 15-PGDH inhibitor to PGE2-expressing cells, or a culture thereof.

### Effects of the Invention

The pharmaceutical composition according to the present invention can have an antiinflammatory effect, an effect of IgE reduction in the serum, an effect of NO and MDA reduction, an inhibitory effect of proliferation of activated T cells, and an effect of improving proliferation ability of skin fibroblasts and epithelial keratinocytes, in a mouse model of DNCB-induced AD.

### BRIEF DESCRIPTION OF THE DRAWINGS

FIG. 1 illustrates a test schedule according to an embodiment of the present invention.

FIG. 2 illustrates the results of visual lesions in mice of DNCB-induced atopy according to days.

FIG. 3 illustrates the results of skin histological analysis in mice DNCB-induced atopy.

FIG. 4 illustrates the concentrations of IgE and TGF-β1 in the serum of mice DNCB-induced atopy.

FIG. 5 illustrates the concentrations of NO and MDA in the serum of mice DNCB-induced atopy.

FIG. 6 illustrates the effect of mesenchymal stem cells on the immunosuppression of activated T cells.

FIG. 7 illustrates the analysis results of immunophenotypes of activated T cells.

FIG. 8 illustrates the analysis results of proliferative capacity of skin fibroblasts and epithelial keratinocytes.

### DESCRIPTION OF EXEMPLARY EMBODIMENTS

1. Pharmaceutical Composition for Immune Diseases and Inflammatory Diseases

According to a first embodiment,

the present invention provides a pharmaceutical composition for the prevention or treatment of immunological diseases or inflammatory diseases, containing cells cultured by adding a 15-hydroxyprostaglandin dehydrogenase (15-PGDH) inhibitor to prostaglandin E2 (PGE2)-expressing cells, or a culture thereof.

According to the pharmaceutical composition of the present invention for the prevention or treatment of immunological diseases or inflammatory diseases, the cell may be human adult stem cells, human pluripotent stem cells, induced pluripotent stem cells, animal embryonic stem cells, or animal adult stem cells. The adult stem cells may be mesenchymal stem cells, mesenchymal stromal cells derived from human tissue, mesenchymal stem cells derived from human tissue, multipotent stem cells, or amniotic epithelial cells. Additionally, the adult stem cells may be mesenchymal stem cells derived from umbilical cord, mesenchymal stem cells derived from umbilical cord blood, mesenchymal stem cells derived from bone marrow, mesenchymal stem cells derived from adipose tissue, mesenchymal stem cells derived from muscle tissue, mesenchymal stem cells derived from neurons, mesenchymal stem cells derived from skin, mesenchymal stem cells derived from amniotic membranes, or mesenchymal stem cells derived from the placenta.

According to the pharmaceutical composition for the prevention or treatment of immunological diseases or inflammatory diseases of the present invention, the immunological diseases or inflammatory diseases may be autoimmune diseases, graft rejection, arthritis, graft versus host disease, bacterial infection, sepsis, or inflammation. The autoimmune disease may be Crohn's disease, erythema, atopy, rheumatoid arthritis, Hashimoto's thyroiditis, malignant anemia, Edison's disease, Type 1 diabetes, lupus, chronic fatigue syndrome, fibromyalgia, hypothyroidism and hyperthyroidism, scleroderma, Behcet's disease, inflammatory bowel disease, multiple sclerosis, myasthenia gravis, Meniere's syndrome, Guilian-Barre syndrome, Sjogren's syndrome, vitiligo, endometriosis, psoriasis, systemic scleroderma, asthma, and ulcerative colitis.

According to a second embodiment,

the present invention provides a method for inhibiting an immune response or inflammatory response in a subject excluding humans, which includes administering, to a subject, cells cultured by adding a 15-PGDH inhibitor to PGE2-expressing cells or a culture thereof.

According to the method for inhibiting an immune response or inflammatory response, the method may perform the prevention or treatment of immunological diseases or inflammatory diseases of a subject by inhibiting an immune response or inflammatory response.

According to the method for inhibiting an immune response or inflammatory response, the administration may be intraperitoneal or intravascular administration, direct administration to the lesion or administration into the synovial cavity of the joint.

According to the method for inhibiting an immune response or inflammatory response, the subject may include cattle, dogs, pigs, chickens, sheep, horses, or all of the animals excluding humans.

According to a third embodiment,

the present invention provides a method for preparing an immunosuppressive agent or antiinflammatory agent, which includes adding a 15-PGDH inhibitor to PGE2-expressing cells followed by culturing the same.

According to the method for preparing an immunosuppressive agent or antiinflammatory agent of the present invention, the concentration of the 15-PGDH inhibitor is in the range of 0.01 μg/mL to 100 μg/mL relative to the medium, and the cultivation time is in the range of 0.1 to 200 hours.

According to a fourth embodiment,

the present invention provides a method for preparing PGE2, which includes culturing PGE2-expressing cells in a medium, to which a 15-PGDH inhibitor was added, and PGE2 or transforming growth factor beta 1 (TGF-β1) is secreted from these cells during cultivation.

According to a fifth embodiment,

the present invention provides an implant which includes PGE2-expressing cells and a 15-PGDH inhibitor.

According to an implant, the PGE2-expressing cells may be cultured along with a 15-PGDH inhibitor and then removed.

According to a sixth embodiment,

the present invention provides a method for preparing an implant which includes culturing by adding a 15-PGDH inhibitor to PGE2-expressing cells.

According to the method for preparing an implant of the present invention, the method may further include removing cells after culturing the cells.

According to a seventh embodiment,

the present invention provides a complex which includes PGE2-expressing cells and a 15-PGDH inhibitor.

According to the complex of the present invention, a 15-PGDH inhibitor may be bound to the PGE2-expressing cells. As the 15-PGDH inhibitor is bound to the PGE2 and thereby the PGE2 may be activated.

According to an eighth embodiment,

the present invention provides a culture obtained by adding a 15-PGDH inhibitor to PGE2-expressing cells.

The pharmaceutical composition according to the present invention for the prevention or treatment of immunological diseases and inflammatory diseases is a cell therapy agent which can replace the existing immunosuppressive agents and inflammation inhibitors known to have side effects and has no side effect and is able to be economically used, and can be effectively used for the prevention or treatment of immunological diseases and inflammatory diseases, such as Crohn's disease, rheumatoid arthritis, atopy, etc.

2. Pharmaceutical Composition for Treating Wounds

The present invention provides a pharmaceutical composition for the prevention or treatment of wounds, containing cells cultured by adding a 15-hydroxyprostaglandin dehydrogenase (15-PGDH) inhibitor to prostaglandin E2 (PGE2)-expressing cells, or a culture thereof.

According to the pharmaceutical composition of the present invention for the prevention or treatment of wounds, the cell may be human adult stem cells, human pluripotent stem cells, induced pluripotent stem cells, animal embryonic stem cells, or animal adult stem cells. The adult stem cells may be mesenchymal stem cells, mesenchymal stromal cells derived from human tissue, mesenchymal stem cells derived from human tissue, multipotent stem cells, or amniotic epithelial cells. Additionally, the adult stem cells may be mesenchymal stem cells derived from umbilical cord, mesenchymal stem cells derived from umbilical cord blood, mesenchymal stein cells derived from bone marrow, mesenchymal stem cells derived from adipose tissue, mesenchymal stem cells derived from muscle tissue, mesenchymal stem cells derived from neurons, mesenchymal stem cells derived from skin, mesenchymal stem cells derived from amniotic membrane, or mesenchymal stem cells derived from the placenta.

According to the pharmaceutical composition of the present invention for the prevention or treatment of wounds, the wounds may include wounds such as contusion or bruise, non-healing traumatic wound, destruction of tissue by irradiation, abrasion, bone gangrene, laceration, avulsion, penetrated wound, gunshot wound, incised wound, burns, frostbite, skin ulcer, dry skin, skin keratosis, crack, rupture, dermatitis, pain due to dermatophytosis, operative wound, vascular disease wound, and corneal wound; conditions related to diabetes and circulatory failure such as pressure sore, bed wound, diabetic skin erosion; chronic ulcer, suture regions after plastic surgery, spinal cord injury, gynecological wound, chemical wound, and acne. Preferably, these wounds may include abrasion, laceration, stab wound, incised wound, avulsion, penetrated wound, or skin ulcer, but the wounds are not limited thereto.

3. Cosmetic Composition

The present invention provides a cosmetic composition containing cells cultured by adding a 15-hydroxyprostaglandin dehydrogenase (15-PGDH) inhibitor to prostaglandin E2 (PGE2)-expressing cells, or a culture thereof.

According to the cosmetic composition of the present invention, the cosmetic composition can be used for the purposes of skin improvement, wrinkle improvement, hair growth, prevention of hair loss, improvement of premature gray hair, etc.

According to the cosmetic composition of the present invention, the cosmetic composition may be in the form of a solution, a lotion, an emulsion in which oil is dispersed in water or water is dispersed in oil, a gel, a lipstick, and an aerosol.

According to the cosmetic composition of the present invention, the cosmetic composition may be prepared by combining the conventionally used additive components such as oils, fats, emulsifiers, humidifiers, humectants, softeners, preservatives, surfactants, thickeners, perfumes, pigments, dyes and alcohols, polyols, electrolytes, silicone derivatives, etc.

Hereinafter, the present invention will be described in more detail with reference to Examples. It should be obvious to those skilled in the art that these Examples are for illustrative purposes only and that the scope of the present invention is not construed as being limited by these Examples.

### EXAMPLES

### Example 1. In Vivo Validity Test

Validity assay was performed on atopic dermatitis, which is a kind of immune disease of adipose derived mesenchymal stem cells (hereinafter, ADSC), using mice in which atopic dermatitis was induced by DNCB.

1-1. Establishment of Animal Model with Atopic Dermatitis

(1) Animals

The experimental animals (5 week-old male BALB/c mice: initial weight 18 g to 20 g) were allowed to adapt in a breeding room for one week for experimental animals and used in the experiment. During the experiment, the animals were given ad libitum access to water, and the breeding room was maintained at an appropriate temperature (22±2° C.) and relative humidity (60±5%) and the illumination (06:00 AM to 18:00 PM) was adjusted to a 12 hour cycle.

(2) DNCB Preparation

DNCB to be used in the primary and secondary sensitization was used after dissolving at concentrations of 1% (primary sensitization) and 0.2% (secondary sensitization) in a mixed solution (acetone:olive oil).

(3) Sensitization Procedure

After removing hair from the remaining experimental groups except the negative control, 150 μL of 1% DNCB was applied on the back of the mice to a diameter of 0.8 cm for a period of 5 days, and after 4 days, 150 μl, of 0.2% DNCB solution was applied for 3 days for secondary sensitization.

1-2. Cultivation of Stem Cells

With regard to the cultivation of ADSC, public IRB approval was obtained to receive fat tissue supply from Dietclinic (located in Seoul, Korea) and ADSC isolation was performed by the following method. First, tissue was broken down using collagenase and only singles cells were isolated using a cell strainer and cultured in a culture flask along with culture medium, and cells were proliferated through continuous subcultures. All of the cells used in this test were from the 5th subculture. Human bone marrow-derived mesenchymal stem cells (hereinafter, BMSC) and human umbilical cord-derived mesenchymal stem cells (hereinafter, UCSC) were purchased from Promocell (Germany) for use.

1-3. Preparation and Treatment of 15-PGDH Inhibitor

15-PGDH inhibitor was offered from the Research Lab of Professor Cho Hoon (Chosun University, Korea) and used at a final concentration of 5 μM. Additionally, each stem cell was treated with a 15-PGDH inhibitor along with culture medium for 4 to 12 hours (priming), and cells washed 3 times with physiological saline were collected and used in the test (ADSC primed with a 15-PGDH inhibitor; PADSC, primed with a 15-PGDH inhibitor; and PBMSC, primed with a 15-PGDH inhibitor), and the culture broth used in the test was treated with the 15-PGDH inhibitor onto each stem cell for 4 to 12 hours, the culture broth was replaced with a fresh culture broth, cultured for 24 hours, and the resulting culture broth was used.

1-4. Administration of ADSC

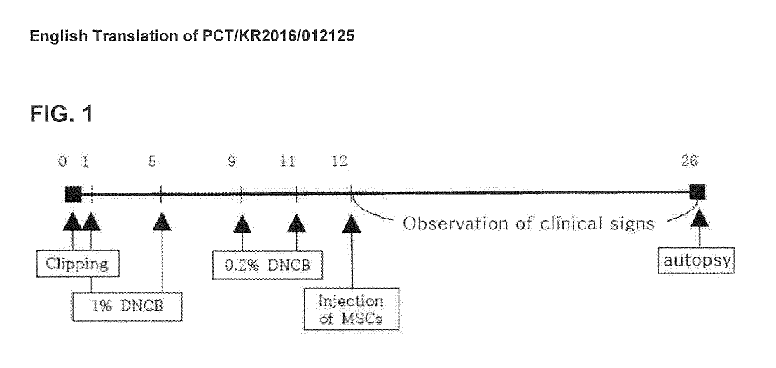

The introduction of ADSC was performed by intravenous injection to a caudal vein of mice, and fibroblast-treated group, which is cell-treated control, and 15-PGDH inhibitor-treated ADSC were also tested in the same manner as ADSC. In addition, with regard to the schedule of administration of ADSC (FIG. 1), a single administration was performed on the 12th day of the test, and visual lesions were confirmed for 2 weeks after administration.

1-5. Separation of Groups

The experimental groups are as follows and they were separated without statistical significance (Table 1). One day after a second sensitization, a new group separation was performed, and cell-treated groups were administered to the caudal vein (1×10⁶ cells/head), and negative control group and DNCB control group were administered with the same dose of physiological saline.

TABLE 1

| Group | Sensitization | Cell Injection | No. of Animals |
|---|---|---|---|
| Negative Control | x | x | 7 |
| DNCB Control | ∘ | x | 7 |
| Fibroblast Control | ∘ | ∘ | 7 |
| ADSC-treated Group | ∘ | ∘ | 7 |
| (ADSC) | | | |
| ADSC-treated Group | ∘ | ∘ | 7 |
| treated with 15-PGDH | | | |
| Inhibitor (PADSC) | | | |

1-6. Evaluation of Visual Lesions

With regard to the evaluation of visual lesions, visual remedial effect on each mouse was determined once daily from the time of group separation. The severity of the disease was assessed by assigning scores for dryness, excoriation, erythema and edema from 0 to 3 (0, none; 1, mild; 2, moderate; and 3, severe) according to symptoms, and the values were summed and evaluated. The values were added every 3 days after the administration, and the significant changes among the groups were compared and analyzed by the mean value of each group.

1-7. Autopsy

An autopsy was performed after observing clinical signs for 2 weeks after ADSC administration.

Blood samples were collected, stored for 4 hours in a refrigerator, and centrifuged to obtain sera. Mouse dorsal skin was cryopreserved (−80° C.) to measure NO/MDA levels and molecular biology assays.

1-8. Skin Biopsy

After 2 weeks of administration, the animals were euthanized and the tissue regions with dermatitis lesions were secured. The tissues were fixed in 4% paraformaldehyde, embedded in paraffin, and subjected to H & E staining and Toluidine blue staining. The changes in the thickness of epithelial layer were analyzed by microscopic observation of the H & E stained skin tissues, and the degree of degranulation of mast cells was continued by Toluidine blue staining.

1-9. IgE, PGE2, TGFb1 Concentrations in Serum

Each of IgE, PGE2, TGFb1 concentrations in serum was measured on a microplate reader using a mouse ELISA kit.

1-10. NO/MDA Levels in Damaged Skin and Plasma

From an immunological point of view, it is known that the expression of NO synthase increases the production of NO and the amount of NO increases at sites showing pathological tissue damage. In addition, the increase of NO in dermal cells promotes the increase of reactive oxygen species that induces lipid peroxidation. NO is a factor which is expressed in a large amount in inflammatory diseases such as allergy and asthma thereby inducing inflammation, and the functional diversity expressed by NO is known to vary depending on the concentration and activation of target cells. The nitric oxide (NO) content was measured by the Griess reaction of NO₂⁺ (nitrite), a stable NO oxide. The tissues were ground with 50 mM potassium phosphate buffer containing EDTA and centrifuged at 10,000×g for 15 min at 4° C. 100 μL Each of the supernatant of the tissues and serum were placed in a 96-well plate, and an equal amount of Griess reagent (0.1% N-1-naphthyl-ethylendiamine/H₂O: 1% sulfanilamide/5% H₃PO₄=1:1) was added thereto and reacted for 10 minutes and the absorbance was measured at 570 nm using a microplate reader. The concentration of nitrite was expressed by comparing with the standard curve obtained using sodium nitrite

When NO generation is induced by the administration of allergen, the lipid peroxidation of cells is induced by NO and the surrounding reactive oxygen species thereby producing an oxidation reactive material such as MDA. MDA is an oxide of polyunsaturated fatty acid by oxidation reaction and is known as evidence of oxidative damage (inflammation) of tissue cells. The content of malondialdehyde (MDA) was measured using thiobarbituric acid (TBA). That is, serum and tissue samples (50 μL) were added to distilled water (1 mL) and 29 mM TBA (1 mL) dissolved in acetic acid, reacted at 95° C. for 1 hour, cooled, and 5 mM HCl (25 μL) was added thereto. Extraction was performed by adding n-butanol (3.5 mL) thereto and the butanol layer was collected by centrifugation at 1,500×g for 5 minutes and then absorbance was measured at 532 nm. At this time, the standard curve was prepared using 1,1,3,3-tetraethoxypropane and the concentration of MDA was expressed in terms of WI/and wet tissue.

1-11. Statistical Processing of Data

One-way ANOVA was performed for the statistical analysis of the data from the experiment, and the significance between the groups was tested at the p=0.05 level. When significance was acknowledged, Dunnett's t-test was performed so as to test statistical significance between the control and test groups (p<0.05).

### Example 2. In Vitro Validity Assay

2-1. Inhibitory Function of Mesenchymal Stem Cells Against Activated T Cells

T cells isolated from human peripheral blood were activated by treatment with various mitogens (anti-CD3, PHA, and IL-2) and co-cultured with each of ADSC, UCSC, BMSC, PADSC, PUCSC, and PBMSC, and upon completion of the culture, the proliferation rate of T cells was measured using an ADAM cell counter. During the co-culture, it was observed whether inhibition dependent on the concentration of mesenchymal stem cells was induced by setting the concentration of each kind of mesenchymal stem cells at 1×10⁴(10:1), 5×10⁴(5:1), 1×10⁵(1:1) relative to T cells, and co-culture was also performed after preventing the cell-contact between T cells and mesenchymal stem cells using transwell plates so as to confirm whether each kind of mesenchymal stem cells inhibit T cells by cell-contact. The number of cells counted by the cell counter was calculated based on the number of cells in terms of % based on the number of activated T cells without co-culture (100% cell count).

2-2. Immunolabeled Expression Analysis of Activated T Cells Co-Cultured with Stem Cells

Mesenchymal stem cells, mesenchymal stem cells treated with a 15-PGDH inhibitor, T cells isolated from human peripheral blood, and culture broth treated with various mitogens were co-cultured in Transwell for 5 days. Upon completion of cultivation, only T cells were collected and T cell surface antibodies were analyzed using flow cytometry (FACS).

1-3. Proliferative Capacity Test Between Skin Fibroblasts and Epithelial Keratinocytes

After treating or untreating ADSC, UCSC, and BMSC with a 15-PGDH inhibitor for 4 to 12 hours, the medium was replaced with a fresh culture broth, in which the 15-PGDH inhibitor was not added, and cultured for 24 hours and the culture broth was collected. One day before the test, 20,000 cells each of skin fibroblasts (HDF) and epithelial keratinocytes (HEK) were inoculated into 6 wells, and on the day of the test, the culture medium was replaced with the culture broth collected from above. Then, the cells were cultured in a 5% CO₂ incubator, and after 3 days, the number of cells was counted using a cell counter. All tests were performed in triplicate and standard deviation values were calculated therefrom.

### Experimental Example

### Experimental Example 1. In Vivo Validity Assay

1-1. Results of Evaluation of Visual Lesions

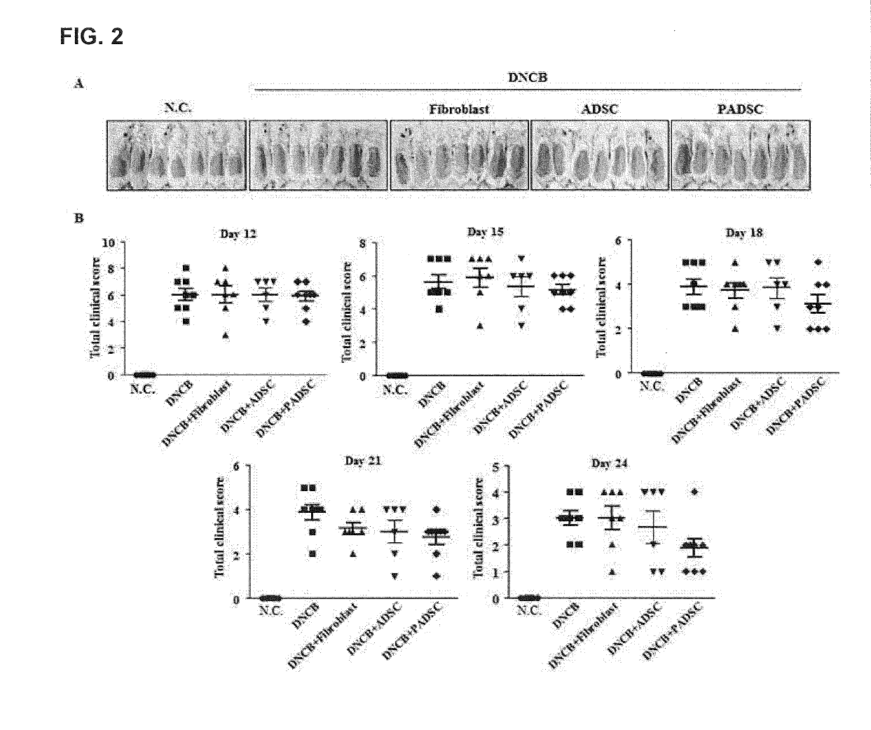

The anti-inflammatory effect of ADSC was observed in a mouse model of DNCB-induced allergic dermatitis (AD) (FIG. 2A). After the MSC administration, dryness, excoriation, erythema, edema, etc. were observed at intervals of 3 days and the severity of the diseases were assessed by assigning scores from 0 to 3 and the values were compared (FIG. 2B).

As a result, it was confirmed that the severity of diseases was reduced from 10 days later (Day 21) compared to the DNCB group when ADSC and PADSC were administered.

1-2. Results of Skin Biopsies

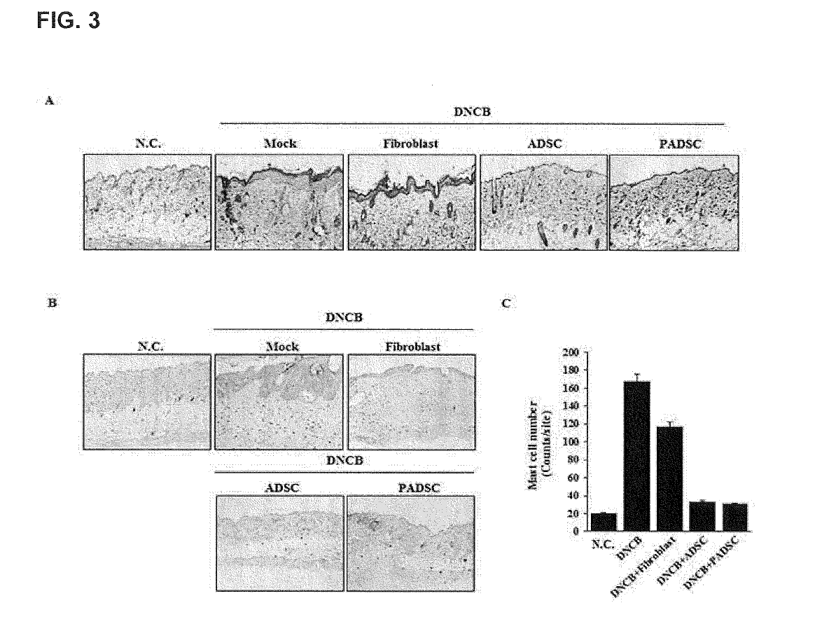

Changes in tissue structure, mast cell infiltration, and degranulation were observed on the back of mice in which the anti-inflammatory effect by ADSC was shown. After the end of the experiment, mouse tissues on the back were extracted and paraffin blocks were prepared, and the tissues were cut to a thickness of 6 μm and stained with H & E and Toluidine blue. H & E staining was performed for each of these tissues (FIG. 3).

As a result, the swelling of the epidermis and dermis was noticeably observed in the DNCB-treated group (control group) compared to the normal mouse (negative control group: NC), and the treatment with ADSC and PADSC compared to the Fibroblast-treated group significantly reduced the swelling of the epidermis and dermis compared to the control group (FIG. 3A). From these results, it was continued that the acute inflammatory response induced by DNCB can be reduced by ADSC and PADSC.

Additionally, as a result of observation of mast cell infiltration and degranulation after staining with Toluidine blue, the group treated with ADSC and PADSC showed a very few number of mast cell infiltration and degranulation compared to the control group (FIG. 3B).

1-3. Results of Analysis of IgE and Major Inflammatory Cytokines

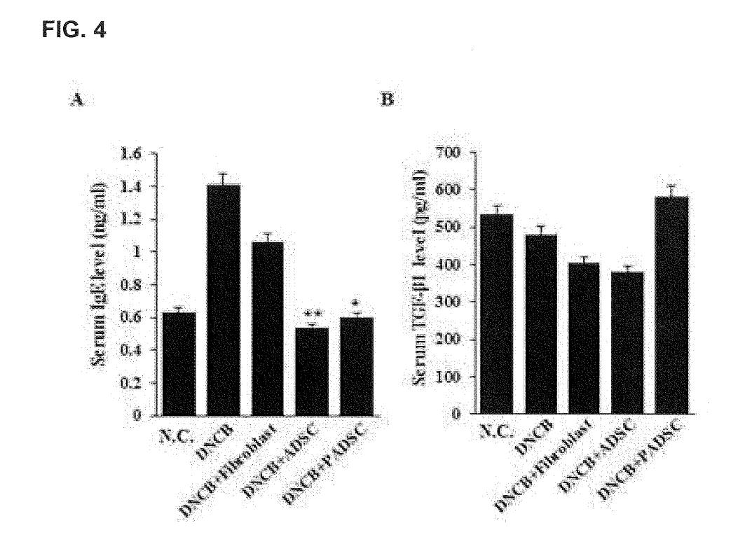

IgE, PGE2, and TGF-β1 levels were measured using the Elisa kit so as to observe changes in inflammatory response markers and inflammatory cytokines (FIG. 4).

As a result, it was confirmed that in the ADSC and PADSC-treated groups, serum IgE levels remained at low levels compared to the control group.

1-4. Results of Analysis of NO and MDA

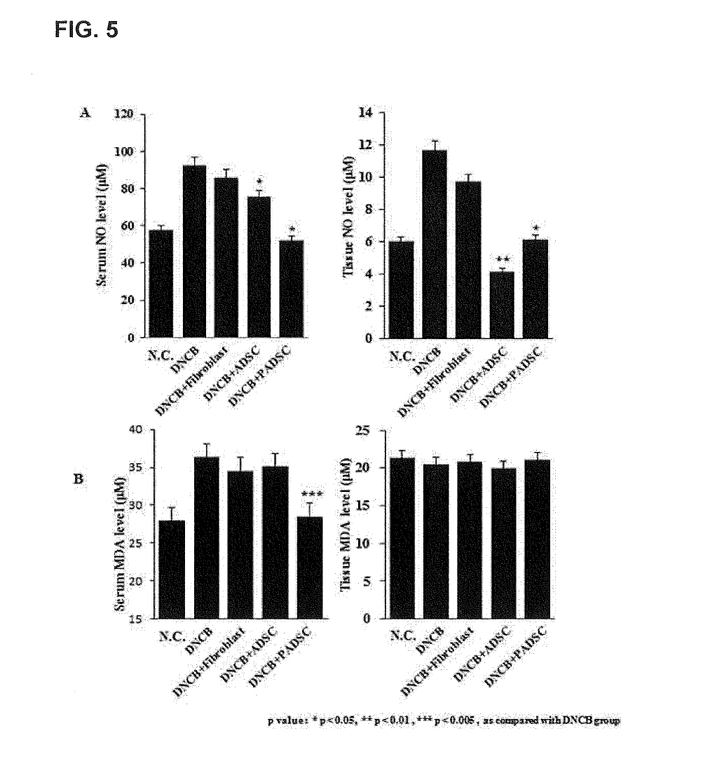

In the case of inflammatory diseases such as allergies and asthma, there occurs generation of nitric oxide (NO), which induces lipid peroxidation by the surrounding reactive oxygen species thus becoming a malondialdehyde (MDA) oxide. Therefore, the contents of NO and MDA were measured in the serum and lesion tissues (FIG. 5).

Serum MDA levels were similar to those of normal mice in the PADSC-treated group, whereas in the ADSC group, serum MDA levels showed no significant difference compared to the negative control group (NC). It was confirmed that there was no significant change in the tissues (FIG. 5B).

### Experimental Example 2. In Vitro Validity Assay

2-1. Results of Analysis of Mesenchymal Stem Cells on Inhibitory Function Against Activated T Cells

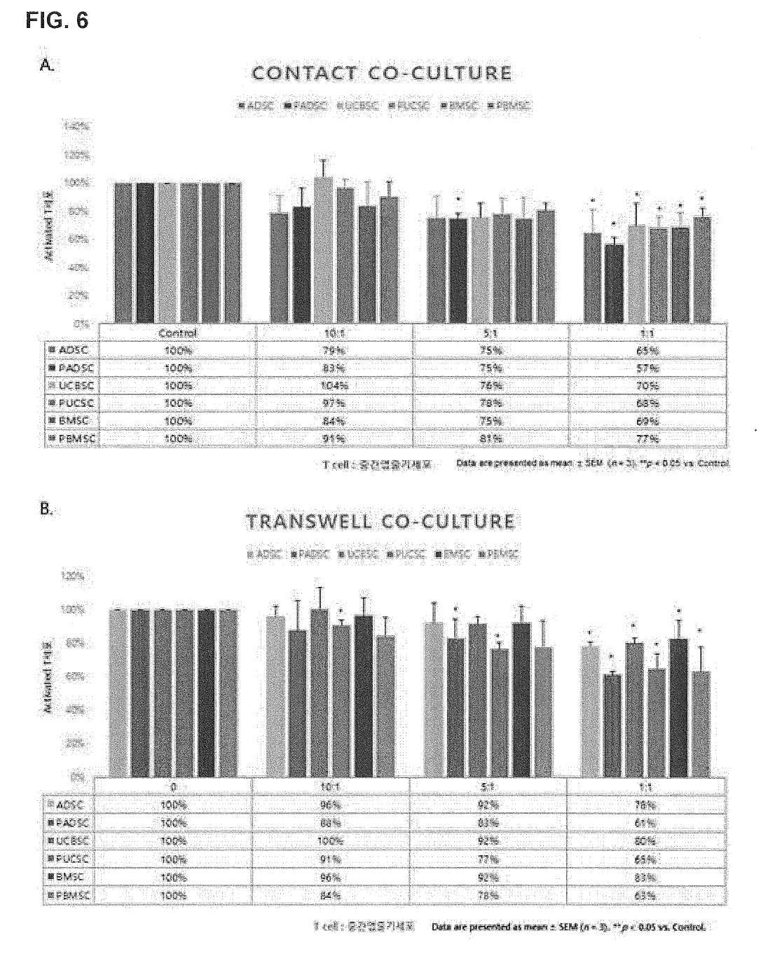

T cells isolated from human peripheral blood with a mitogen were co-cultured with mesenchymal stem cells and 15-PGDH-treated mesenchymal stem cells, and the number of activated T cells was measured (FIG. 6).

As a result, during the co-culture, when mesenchymal stem cells and activated T cells were directly adhered and contact co-cultured, mesenchymal stem cells significantly inhibited the proliferation of activated T cells in an environment where the ratio of T cells to mesenchymal stem cells was 1:1, whereas in the cases of ADSC and UCSC, 15-PGDH-treated mesenchymal stem cells were shown to further inhibit the proliferation of activated T cells (FIG. 6A). Additionally, during the co-culture, when mesenchymal stem cells and activated T cells were co-cultured by separating using an insert not to be in direct contact with each other, mesenchymal stem cells significantly inhibited the proliferation of activated T cells, in an environment where the ratio of T cells to mesenchymal stem cells was 1:1, all of the mesenchymal stem cells treated with 15-PGDH were shown to further inhibit the proliferation of activated T cells (FIG. 6B).

2-2. Analysis Results of Immunolabeled Expression of Activated T Cells

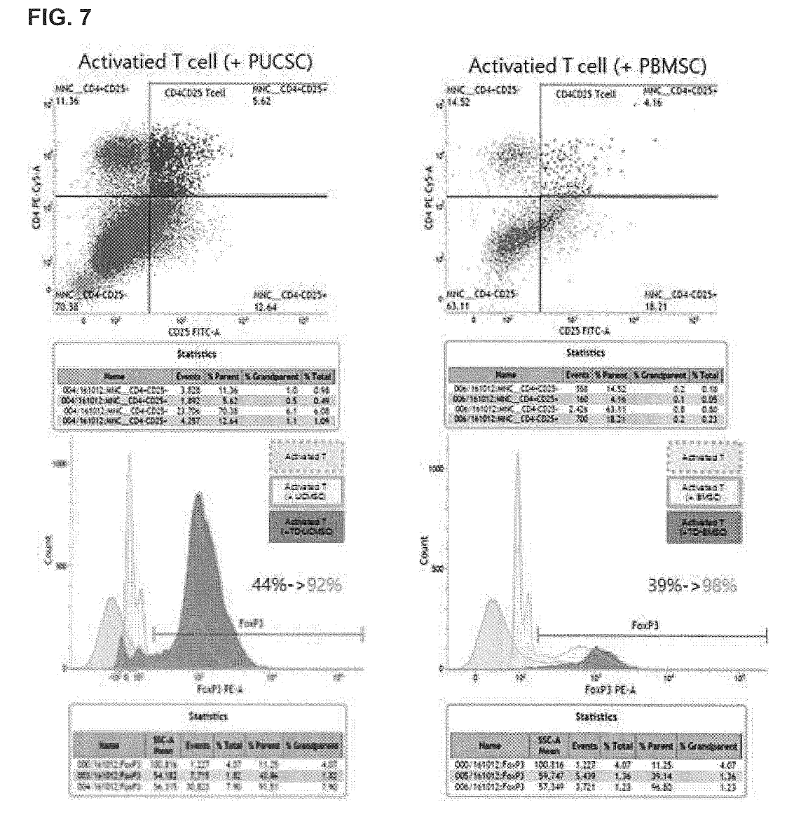

T cells isolated from human peripheral blood with a mitogen were co-cultured with mesenchymal stem cells and 15-PGDH-treated mesenchymal stem cells, and T cells co-cultured with mesenchymal stem cells and 15-PGDH inhibitor-treated mesenchymal stem cells, respectively, were collected and flow cytometry was performed. In particular, the antigens of CD4, CD25, and FoxP3, which are phenotypes of regulatory T cells, were analyzed (FIG. 7).

As a result, the T cells co-cultured with umbilical cord stem cells treated with a 15-PGDH inhibitor (PUCSC) showed about a 48% increase in regulatory T cells compared to the T cells co-cultured with umbilical cord stem cells not treated with a 15-PGDH inhibitor (UCSC)(44%), thus showing a 92% Treg population. Additionally, the T cells co-cultured with bone marrow stem cells (BMSC) showed a 39% Treg population while the T cells co-cultured with bone marrow stem cells not treated with a 15-PGDH inhibitor (PBMSC) showed an increase up to a 98% Treg population. From these results, it was confirmed that mesenchymal stem cells can inhibit activated T cells while increasing regulatory T cells, and 15-PGDH inhibitors can further strengthen the capability of stem cells that enable immunomodulation.

2-3. Results of Proliferative Capacity Test Between Skin Fibroblasts and Epithelial Keratinocytes

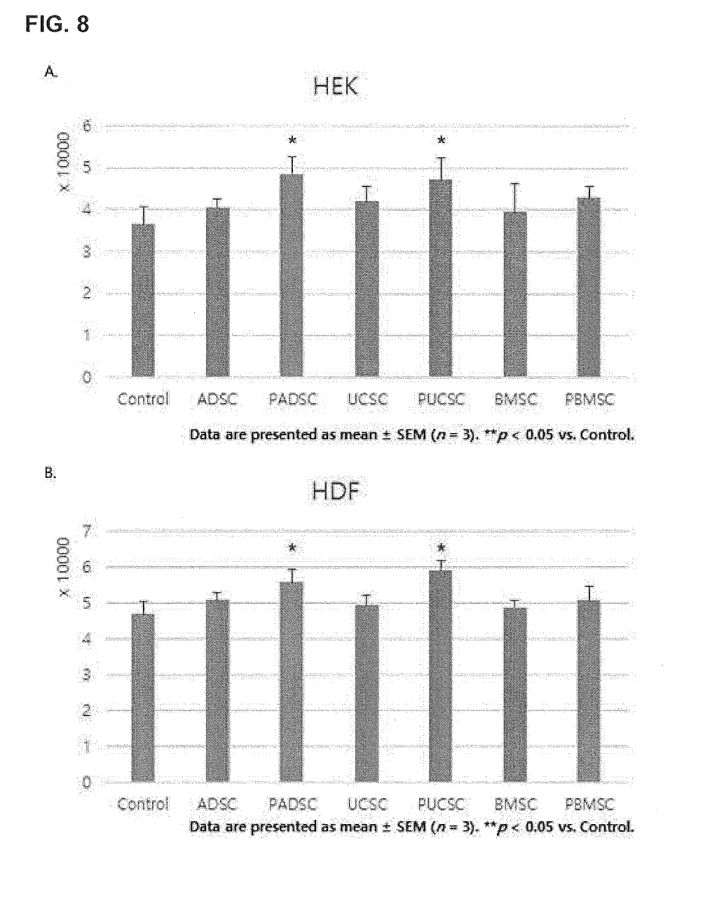

PADSC, PUCSC, and PBMSC, which were primed with ADSC, UCSC, and BMSC, were cultured for 24 hours, and each culture was collected, and the growth rates of human skin fibroblasts (HDF) and human epithelial keratinocytes (HEK) were compared (FIG. 8).

As a result, in the case of HDF, the growth rate significantly increased when cultured with PADSC and PUCSC culture broths primed with a 15-PGDH inhibitor, compared to the control group of the culture broth. Likewise, in the case of HEK, it was confirmed that the growth rate increases when cultured with PADSC and PUCSC culture broths primed with a 15-PGDH inhibitor, compared to the control group of the culture broth.

Those skilled in the art to which the present invention belongs will be able to understand that the present invention may be embodied in various other forms without departing from the spirit or essential characteristics thereof. Therefore, the disclosed Examples should be considered from an illustrative point of view, not from a restrictive point of view. The scope of the present invention is shown in claims not in the explanations described above and it should be interpreted that all of the modifications and variations within an equivalent range thereof are included in the present invention.

The pharmaceutical composition according to the present invention can have an antiinflammatory effect, an effect of IgE reduction in the serum, an effect of reducing NO and MDA, an inhibitory effect of proliferation of activated T cells, and an effect of improving proliferative capacity of skin fibroblasts and epithelial keratinocytes, in a mouse model of DNCB-induced AD. Accordingly, the pharmaceutical composition according to the present invention is expected to be effectively used as an immunosuppressive agent, an antiinflammatory agent, or a wound healing agent.

1-24. (canceled)

25. A pharmaceutical composition for the prevention or treatment of immunological diseases, inflammatory diseases, or wounds, comprising cells cultured by adding a 15-hydroxyprostaglandin dehydrogenase (15-PGDH) inhibitor to prostaglandin E2 (PGE2)-expressing cells, or a culture thereof.

26. The pharmaceutical composition of claim 25, wherein the 15-PGDH inhibitor is at least one kind selected from the group consisting of cyclooxygenase inhibitors, flavonoids, phytophenolic compounds, and antagonists of peroxisome proliferator-activated receptor gamma (PPARγ).

27. The pharmaceutical composition of claim 25, wherein the cells are stem cells.

28. The pharmaceutical composition of claim 25, wherein the immunological diseases or inflammatory diseases are autoimmune diseases, graft rejection, arthritis, graft versus host disease, bacterial infection, sepsis, or inflammation, and
the autoimmune disease is at least one kind selected from the group consisting of Crohn's disease, erythema, atopy, rheumatoid arthritis, Hashimoto's thyroiditis, malignant anemia, Edison's disease, Type 1 diabetes, lupus, chronic fatigue syndrome, fibromyalgia, hypothyroidism and hyperthyroidism, scleroderma, Behcet's disease, inflammatory bowel disease, multiple sclerosis, myasthenia gravis, Meniere's syndrome, Guilian-Barre syndrome, Sjogren's syndrome, vitiligo, endometriosis, psoriasis, systemic scleroderma, asthma, and ulcerative colitis.

29. A method for inhibiting an immune response, an inflammatory response, or wounds in a subject excluding humans, comprising administering, to a subject, cells cultured by adding a 15-hydroxyprostaglandin dehydrogenase (15-PGDH) inhibitor to prostaglandin E2 (PGE2)-expressing cells or a culture thereof.

30. The method of claim 29, wherein the administration is intraperitoneal or intravascular administration, direct administration to the lesion or administration into the synovial cavity of the joint.

31. A method for preparing an immunosuppressive agent, an antiinflammatory agent, or a wound healing agent, comprising adding a 15-hydroxyprostaglandin dehydrogenase (15-PGDH) inhibitor to prostaglandin E2 (PGE2)-expressing cells followed by culturing the same.

32. The method according to claim 29, wherein the 15-PGDH inhibitor is at least one kind selected from the group consisting of cyclooxygenase inhibitors, flavonoids, phytophenolic compounds, and antagonists of peroxisome proliferator-activated receptor gamma (PPAR γ).

33. The method according to claim 29, wherein the cells are stem cells.

34. An implant comprising prostaglandin E2 (PGE2)-expressing cells and a 15-hydroxyprostaglandin dehydrogenase (15-PGDH) inhibitor.

35. An implant, which was cultured in prostaglandin E2 (PGE2)-expressing cells by adding a 15-hydroxyprostaglandin dehydrogenase (15-PGDH) inhibitor thereto and then the cells were removed therefrom.

36. The implant of claim 34, wherein the 15-PGDH inhibitor is at least one kind selected from the group consisting of cyclooxygenase inhibitors, flavonoids, phytophenolic compounds, and antagonists of peroxisome proliferator-activated receptor gamma (PPAR γ).

37. A method for preparing an implant comprising culturing by adding a 15-hydroxyprostaglandin dehydrogenase (15-PGDH) inhibitor to prostaglandin E2 (PGE2)-expressing cells.

38. The method of claim 37, further comprising removing cells after cultivation.

39. The method of claim 37, wherein the 15-PGDH inhibitor is at least one kind selected from the group consisting of cyclooxygenase inhibitors, flavonoids, phytophenolic compounds, and antagonists of peroxisome proliferator-activated receptor gamma (PPAR γ).

40. A complex comprising prostaglandin E2 (PGE2)-expressing cells and a 15-hydroxyprostaglandin dehydrogenase (15-PGDH) inhibitor.

41. The complex of claim 40, wherein a 15-PGDH inhibitor is bound to the PGE2 of the cells.

42. The complex of claim 40, wherein a 15-PGDH inhibitor is bound to the PGE2 of the cells and thereby the PGE2 is activated.

43. The method according to claim 31, wherein the 15-PGDH inhibitor is at least one kind selected from the group consisting of cyclooxygenase inhibitors, flavonoids, phytophenolic compounds, and antagonists of peroxisome proliferator-activated receptor gamma (PPAR γ).

44. The method according to claim 31, wherein the cells are stem cells.
